(19) United States
(12) Patent Application Publication
(10) Pub. No.: US 2024/0108028 A1
(43) Pub. Date: Apr. 4, 2024

(54) METHOD FOR PREPARING PROTEIN NANOPARTICLE MICROSPHERES

(71) Applicant: Jiangnan University, Wuxi (CN)

(72) Inventors: Tao WANG, Wuxi (CN); Chen LIN, Wuxi (CN); Zilong CHEN, Wuxi (CN); Ren WANG, Wuxi (CN); Wei FENG, Wuxi (CN); Zhengxing CHEN, Wuxi (CN)

(21) Appl. No.: 18/075,454

(22) Filed: Dec. 6, 2022

(30) Foreign Application Priority Data
Sep. 30, 2022 (CN) 202211214459.2

(51) Int. Cl. A23J 3/24; A23J 3/28

(52) U.S. Cl. CPC A23J 3/24; A23J 3/28; B82Y 30/00

(57) ABSTRACT

A method for preparing protein nanoparticle microspheres includes: dispersing a protein powder in water, adjusting a pH of a solution of the protein powder to between 10.0 and 12.0, stirring, centrifuging, and collecting a first upper dispersion; adjusting the first upper dispersion to be neutral using a cation exchange resin, centrifuging, to yield a protein nanoparticle dispersion; adding eugenol to the protein nanoparticle dispersion, stirring, centrifuging, and removing a precipitant, to yield a core-shell nanoprotein dispersion comprising a eugenol core and a protein shell; and adding the core-shell nanoprotein dispersion to ultrapure water, dialyzing, centrifuging, collecting a second upper dispersion, freeze-drying the second upper dispersion, to yield protein nanoparticle microspheres.

### CROSS-REFERENCE TO RELATED APPLICATIONS

Pursuant to 35 U.S.C.§ 119 and the Paris Convention Treaty, this application claims foreign priority to Chinese Patent Application No. 202211214459.2 filed Sep. 30, 2022, the contents of which, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, MA 02142.

### BACKGROUND

The disclosure related to structure control of food products, and more particularly, to a method for preparing protein nanoparticle microspheres.

Hollow nanoparticles have extensive applications in the fields of food, cosmetics and pharmaceuticals due to their high specific surface area, high load and controllable release characteristics, biocompatibility, and biodegradability.

Conventional hollow nanoparticles are mainly prepared through sacrificial template method. The method is difficult to operate, and includes a template removal process involving irritating chemicals, such as corrosive, toxic, strong acid and strong alkaline additives, so the method is unpopular in the field of food and biomedicine.

### SUMMARY

One objective of the disclosure is to provide a method for preparing protein nanoparticle microspheres. The method involves no organic solvents, so it is environmentally friendly, and the prepared microspheres are biocompatible and biodegradable.

The protein nanoparticles are prepared by cation exchange resin and comprise a hydrophobic core and a hydrophilic shell. Through the hydrophobic protein-ligand binding reaction, the method of the disclosure uses the asymmetric cross diffusion couple of eugenol and protein nanoparticles to induce the centripetal diffusion of eugenol relative to protein nanoparticles, thereby constructing a core-shell nanoprotein dispersion comprising a eugenol core and a protein shell; further, through osmosis, eugenol is removed by dialysis, which forms protein nanoparticles with hollow structure. The method makes use of salting-out effect to induce the hydrophobic aggregation of protein to form a dimension enlarged structure with different internal density to regulate the protein particles, involves nano engraving based on Kirkendall effect and eugenol removal, and finally prepares protein nanoparticles microspheres with controllable hollow structure and controllable particle size, comprising: full-hollow, semi-hollow, and solid-like nanoparticles microspheres.

Specifically, the method for preparing protein nanoparticle microspheres comprises:
1) dispersing a protein powder in water, adjusting a pH of a solution of the protein powder to between 10.0 and 12.0, stirring, centrifuging, and collecting a first upper dispersion;
2) adjusting the first upper dispersion in 1) to be neutral using a cation exchange resin, centrifuging, to yield a protein nanoparticle dispersion;
3) adding eugenol to the protein nanoparticle dispersion, stirring, centrifuging, and removing a precipitant, to yield a core-shell nanoprotein dispersion comprising a eugenol core and a protein shell; and
4) adding the core-shell nanoprotein dispersion to ultrapure water, dialyzing, centrifuging, collecting a second upper dispersion, freeze-drying the second upper dispersion, to yield protein nanoparticle microspheres.

In a class of this embodiment, the protein powder is rice protein powder, cheese egg white powder, soybean protein powder, or a mixture thereof.

In a class of this embodiment, in 1), the pH of the solution of the protein powder is adjusted using 1-5 mol/L NaOH.

In a class of this embodiment, in 1), the solution is centrifuged at 10,000-15,000 g for 10-15 min.

In a class of this embodiment, in 2), the cation exchange resin is macroporous strongly acidic styrene type cation exchange resin, a hydrogen type macroporous weakly acidic acrylic acid type cation exchange resin, or a mixture thereof.

In a class of this embodiment, in 2), the first upper dispersion is centrifuged at 10,000-15,000 g for 10-15 min.

In a class of this embodiment, following 2) and before adding eugenol to the protein nanoparticle dispersion, the method further comprises: adding sodium chloride to the protein nanoparticle dispersion, to induce an aggregation of protein nanoparticles; centrifuging the protein nanoparticle dispersion comprising aggregated protein nanoparticles, and removing the precipitant, to yield a dimension-enlarged protein nanoparticle dispersion.

In a class of this embodiment, a final concentration of sodium chloride in the protein nanoparticle dispersion is 5-10 μmol/L.

In a class of this embodiment, in 3), the eugenol accounts for 1% by volume of the protein nanoparticle dispersion.

In a class of this embodiment, a mass-volume ratio of the protein powder to the eugenol is 0.1:100 g/sL.

In a class of this embodiment, the dimension-enlarged protein nanoparticles have an average particle size of 30-60 nm.

In a class of this embodiment, in 4), a dialysis time is 12-24 hours.

In a class of this embodiment, in 1), a mass-volume ratio of the protein powder to the water is between 1:50 and 1:100 g/mL

In a class of this embodiment, in 4), when a dialysis time is 24 hours, full-hollow protein nanoparticle microspheres are obtained; when a dialysis time is 18 hours, semi-hollow protein nanoparticle microspheres are obtained; and when a dialysis time is 12 hours, solid-like protein nanoparticle microspheres are obtained.

The disclosure also provides the protein nanoparticle microspheres prepared according to the abovementioned method. The protein nanoparticle microspheres are full-hollow, semi-hollow, or solid-like.

The disclosure further provides a use of the protein nanoparticle microspheres in preparing nutrients and drug carriers.

The following advantages are associated with the method for preparing protein nanoparticle microspheres of the disclosure.
1. The method is green and efficient, involving in no organic solvents, and the prepared hollow structure is biocompatible, and its structure size can be adjusted as needed, which is conducive to controlling the degradation rate, nutrient/drug loading rate and nutrient/drug release rate, with potential applications in the field of nutrient/drug carriers.
2. For the first time, the method of the disclosure uses the asymmetric cross diffusion couple of eugenol and protein nanoparticles to induce the centripetal diffusion of eugenol relative to protein nanoparticles, thereby constructing a core-shell nanoprotein dispersion comprising a eugenol core and a protein shell; further, through osmosis, eugenol is removed by dialysis, which forms protein nanoparticles with hollow structure-nano engraving.
3. The method makes use of salting-out effect to induce the hydrophobic aggregation of protein to form a dimension enlarged structure with different internal density to regulate the protein particles, and finally prepares protein nanoparticles microspheres with controllable hollow structure and controllable particle size, with wide application scope and value.

### BRIEF DESCRIPTION OF THE DRAWINGS

FIG. 1 is a structural diagram and particle size distribution of rice protein nanoparticles of Example 1 of the disclosure;

FIG. 2 is a particle size distribution of dimension-enlarged rice protein nanoparticles of Example 4 of the disclosure;

FIG. 3 is a scatter diagram of a core-shell rice nanoprotein of Example 1 of the disclosure;

FIG. 4 is a structural diagram of protein nanoparticle microspheres prepared in Examples 1-3; pictures 1 and 4 are a scanning electron microscope (SEM) image and a transmission electron microscope (TEM) image of full-hollow protein nanoparticle microspheres of Example 1; pictures 2 and 5 are a scanning electron microscope (SEM) image and a transmission electron microscope (TEM) image of semi-hollow protein nanoparticle microspheres of Example 2; and pictures 3 and 6 are a scanning electron microscope (SEM) image and a transmission electron microscope (TEM) image of solid-like protein nanoparticle microspheres of Example 3;

FIG. 5 is structural diagrams of rice protein nanoparticle dispersion treated by different concentrations of eugenol in Example 6; picture a is treated by 0.4% eugenol; picture a is treated by 0.6% eugenol; and picture a is treated by 1% eugenol;

FIG. 6 is structural diagrams of rice protein nanoparticle microspheres treated by different concentrations of eugenol in Example 6; picture a is treated by 0.4% eugenol; picture a is treated by 0.6% eugenol; and picture a is treated by 1% eugenol;

FIG. 7 is a structural diagram of rice protein prepared in comparative example 1; and

FIG. 8 is a structural diagram of rice protein prepared in comparative example 2.

### DETAILED DESCRIPTION OF THE EMBODIMENTS

To further illustrate the disclosure, embodiments detailing a method for preparing protein nanoparticle microspheres are described below. It should be noted that the following embodiments are intended to describe and not to limit the disclosure.

The hydrogen type macroporous weakly acidic acrylic cation exchange resin in the examples refers to the ion exchange resin of a perforated acrylic copolymer with a carboxylic acid group (—COOH), which was purchased from Bioengineering Co., Ltd.

Eugenol was purchased from Sigma company.

### Example 1

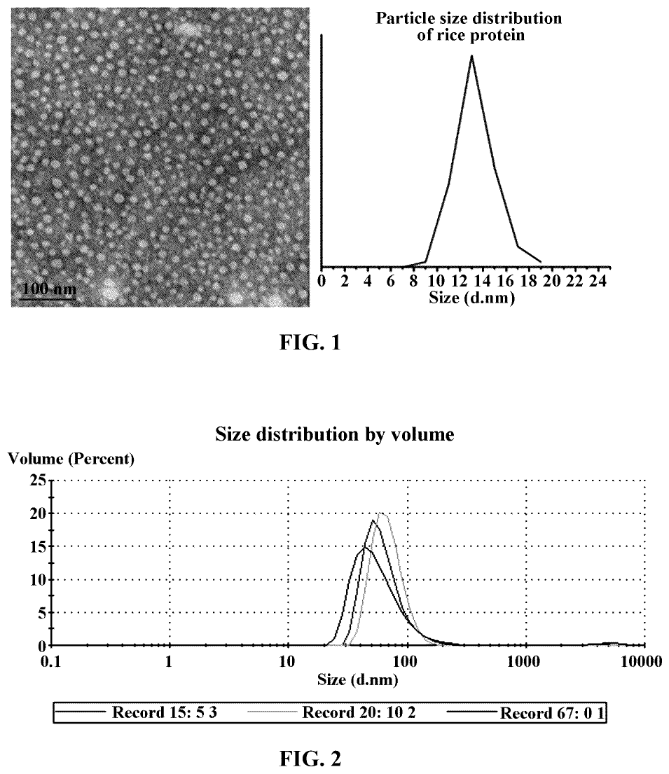

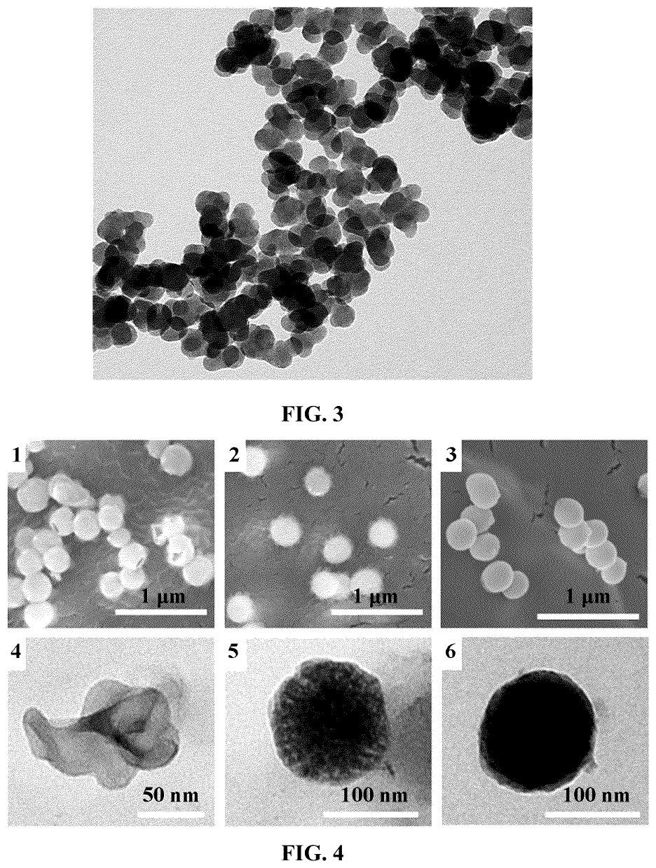

A method for preparing protein nanoparticle microspheres with adjustable hollow structure was detailed as follows:
(1) Structural unfolding of proteins: 0.1 g of rice protein powders were dispersed in ultrapure water in a mass-volume ratio of 1:100 g/mL. The pH of the rice protein solution was adjusted to 10.0 with 4 mol/L NaOH solution. The mixed solution was stirred completely for the structural unfolding of proteins, and centrifuged at 10,000 g for 10 min. The supernatant was collected and precipitant removed.
(2) Ion exchange: The pH of the supernatant obtained in (1) was adjusted using hydrogen type macroporous weakly acidic acrylic cation exchange resin to 7.0, and centrifuged at 10,000 g for 10 min. The precipitant was removed, and a uniform rice protein nanoparticle dispersion having an average particle size of 20 nm was obtained (as shown in FIG. 1).
(3) Core-shell structure development: 1% of eugenol (based on the volume of the rice protein nanoparticle dispersion) was added to the rice protein nanoparticle dispersion. The mixture was stirred completely, and centrifuged at 4,000 g for 10 min. The precipitant was removed, and a core-shell nanoprotein dispersion comprising a eugenol core and a rice protein shell was obtained (as shown in FIG. 3).
(4) Hollow structure regulation: The core-shell nanoprotein dispersion obtained in (3) was dialyzed in ultrapure water for 24 hours, and centrifuged at 10,000 g for 10 min. The precipitant was removed, and the upper dispersion was first cooled in a −20° C. freezer for 2 hours, and then stored overnight in a −80° C. freezer, freeze dried for 72 hours, to yield full-hollow protein nanoparticle microspheres having an average particle size of about 100 nm in the liquid state (as shown in pictures 1 and 4 of FIG. 4).

### Example 2

A method for preparing protein nanoparticle microspheres with adjustable hollow structure was detailed as follows:
(1) Structural unfolding of proteins: 0.1 g of rice protein powders were dispersed in ultrapure water in a mass-volume ratio of 1:100 g/mL. The pH of the rice protein solution was adjusted to 10.0 with 4 mol/L NaOH solution. The mixed solution was stirred completely for the structural unfolding of proteins, and centrifuged at 10,000 g for 10 min. The supernatant was collected and precipitant removed.
(2) Ion exchange: The pH of the supernatant obtained in (1) was adjusted using hydrogen type macroporous weakly acidic acrylic cation exchange resin to 7.0, and centrifuged at 10,000 g for 10 min. The precipitant was removed, and a uniform rice protein nanoparticle dispersion having an average particle size of 20 nm was obtained.
(3) Core-shell structure development: 1% of eugenol (based on the volume of the rice protein nanoparticle dispersion) was added to the rice protein nanoparticle dispersion. The mixture was stirred completely, and centrifuged at 4,000 g for 10 min. The precipitant was removed, and a core-shell nanoprotein dispersion comprising a eugenol core and a rice protein shell was obtained.
(4) Hollow structure regulation: The core-shell nanoprotein dispersion obtained in (3) was dialyzed in ultrapure water for 18 hours, and centrifuged at 10,000 g for 10 min. The precipitant was removed, and the upper dispersion was first cooled in a −20° C. freezer for 2 hours, and then stored overnight in a −80° C. freezer, freeze dried for 72 hours, to yield semi-hollow protein nanoparticle microspheres having an average particle size of about 100 nm in the liquid state (as shown in pictures 2 and 5 of FIG. 4).

### Example 3

A method for preparing protein nanoparticle microspheres with adjustable hollow structure was detailed as follows:
(1) Structural unfolding of proteins: 0.1 g of rice protein powders were dispersed in ultrapure water in a mass-volume ratio of 1:100 g/mL. The pH of the rice protein solution was adjusted to 10.0 with 4 mol/L NaOH solution. The mixed solution was stirred completely for the structural unfolding of proteins, and centrifuged at 10,000 g for 10 min. The supernatant was collected and precipitant removed.
(2) Ion exchange: The pH of the supernatant obtained in (1) was adjusted using hydrogen type macroporous weakly acidic acrylic cation exchange resin to 7.0, and centrifuged at 10,000 g for 10 min. The precipitant was removed, and a uniform rice protein nanoparticle dispersion having an average particle size of 20 nm was obtained.
(3) Core-shell structure development: 1% of eugenol (based on the volume of the rice protein nanoparticle dispersion) was added to the rice protein nanoparticle dispersion. The mixture was stirred completely, and centrifuged at 4,000 g for 10 min. The precipitant was removed, and a core-shell nanoprotein dispersion comprising a eugenol core and a rice protein shell was obtained.
(4) Hollow structure regulation: The core-shell nanoprotein dispersion obtained in (3) was dialyzed in ultrapure water for 12 hours, and centrifuged at 10,000 g for 10 min. The precipitant was removed, and the upper dispersion was first cooled in a −20° C. freezer for 2 hours, and then stored overnight in a −80° C. freezer, freeze dried for 72 hours, to yield solid-like protein nanoparticle microspheres having an average particle size of about 100 nm in the liquid state (as shown in pictures 3 and 6 of FIG. 4).

### Example 4

A method for preparing protein nanoparticle microspheres with adjustable hollow structure was detailed as follows:
(1) Structural unfolding of proteins: 0.1 g of rice protein powders were dispersed in ultrapure water in a mass-volume ratio of 1:100 g/mL. The pH of the rice protein solution was adjusted to 10.0 with 4 mol/L NaOH solution. The mixed solution was stirred completely for the structural unfolding of proteins, and centrifuged at 10,000 g for 10 min. The supernatant was collected and precipitant removed.
(2) Ion exchange: The pH of the supernatant obtained in (1) was adjusted using hydrogen type macroporous weakly acidic acrylic cation exchange resin to 7.0, and centrifuged at 10,000 g for 10 min. The precipitant was removed, and a uniform rice protein nanoparticle dispersion having an average particle size of 20 nm was obtained.
(3) Dimension enlargement of nanoparticles: sodium chloride was added to the protein nanoparticle dispersion obtained in (2), to induce the aggregation of rice protein nanoparticles, and the concentration of the sodium chloride was 5 μmol/L; the protein nanoparticle dispersion comprising aggregated protein nanoparticles was centrifuged at 10,000 g for 10 min., and the precipitant was removed, to yield a dimension-enlarged rice protein nanoparticle dispersion having an average particle size of 50 nm (as shown in FIG. 2).
(4) Core-shell structure development: 1% of eugenol (based on the volume of the dimension-enlarged rice protein nanoparticle dispersion) was added to the dimension-enlarged rice protein nanoparticle dispersion. The mixture was stirred completely, and centrifuged at 4,000 g for 10 min. The precipitant was removed, and a core-shell nanoprotein dispersion comprising a eugenol core and a rice protein shell was obtained.
(5) Hollow structure regulation: The core-shell nanoprotein dispersion obtained in (4) was dialyzed in ultrapure water for 24 hours, and centrifuged at 10,000 g for 10 min. The precipitant was removed, and the upper dispersion was first cooled in a −20° C. freezer for 2 hours, and then stored overnight in a −80° C. freezer, freeze dried for 72 hours, to yield full-hollow protein nanoparticle microspheres.

### Example 5

A method for preparing protein nanoparticle microspheres with adjustable hollow structure was detailed as follows:
(1) Structural unfolding of proteins: 0.1 g of rice protein powders were dispersed in ultrapure water in a mass-volume ratio of 1:100 g/mL. The pH of the rice protein solution was adjusted to 10.0 with 4 mol/L NaOH solution. The mixed solution was stirred completely for the structural unfolding of proteins, and centrifuged at 10,000 g for 10 min. The supernatant was collected and precipitant removed.
(2) Ion exchange: The pH of the supernatant obtained in (1) was adjusted using hydrogen type macroporous weakly acidic acrylic cation exchange resin to 7.0, and centrifuged at 10,000 g for 10 min. The precipitant was removed, and a uniform rice protein nanoparticle dispersion having an average particle size of 20 nm was obtained.
(3) Dimension enlargement of nanoparticles: sodium chloride was added to the protein nanoparticle dispersion obtained in (2), to induce the aggregation of rice protein nanoparticles, and the concentration of the sodium chloride was 10 μmol/L; the protein nanoparticle dispersion comprising aggregated protein nanoparticles was centrifuged at 10,000 g for 10 min., and the precipitant was removed, to yield a dimension-enlarged rice protein nanoparticle dispersion having an average particle size of 70 nm (as shown in FIG. 2).
(4) Core-shell structure development: 1% of eugenol (based on the volume of the dimension-enlarged rice protein nanoparticle dispersion) was added to the dimension-enlarged rice protein nanoparticle dispersion. The mixture was stirred completely, and centrifuged at 4,000 g for 10 min. The precipitant was removed, and a core-shell nanoprotein dispersion comprising a eugenol core and a rice protein shell was obtained.
(5) Hollow structure regulation: The core-shell nanoprotein dispersion obtained in (4) was dialyzed in ultrapure water for 24 hours, and centrifuged at 10,000 g for 10 min. The precipitant was removed, and the upper dispersion was first cooled in a −20° C. freezer for 2 hours, and then stored overnight in a −80° C. freezer, freeze dried for 72 hours, to yield full-hollow protein nanoparticle microspheres.

### Example 6

Optimization of Usage of Eugenol

A method for preparing protein nanoparticle microspheres with adjustable hollow structure was detailed as follows:
(1) Structural unfolding of proteins: 0.1 g of rice protein powders were dispersed in ultrapure water in a mass-volume ratio of 1:100 g/mL. The pH of the rice protein solution was adjusted to 10.0 with 4 mol/L NaOH solution. The mixed solution was stirred completely for the structural unfolding of proteins, and centrifuged at 10,000 g for 10 min. The supernatant was collected and precipitant removed.
(2) Ion exchange: The pH of the supernatant obtained in (1) was adjusted using hydrogen type macroporous weakly acidic acrylic cation exchange resin to 7.0, and centrifuged at 10,000 g for 10 min. The precipitant was removed, and a uniform rice protein nanoparticle dispersion having an average particle size of 20 nm was obtained.
(3) Core-shell structure development: 0.4%, 0.6%, and 1% of eugenol (based on the volume of the rice protein nanoparticle dispersion) was respectively added to the rice protein nanoparticle dispersion. The mixture was stirred completely, and centrifuged at 4,000 g for 10 min. The precipitant was removed, and a core-shell nanoprotein dispersion comprising a eugenol core and a rice protein shell was obtained.
(4) Hollow structure regulation: The core-shell nanoprotein dispersion obtained in (3) was dialyzed in ultrapure water for 24 hours, and centrifuged at 10,000 g for 10 min. The precipitant was removed, and the upper dispersion was first cooled in a −20° C. freezer for 2 hours, and then stored overnight in a −80° C. freezer, freeze dried for 72 hours, to yield solid-like protein nanoparticle microspheres.

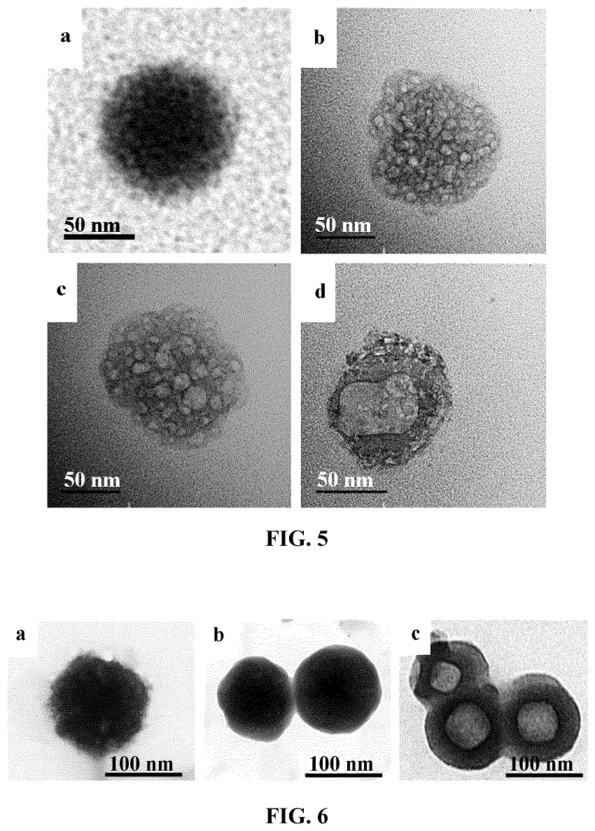

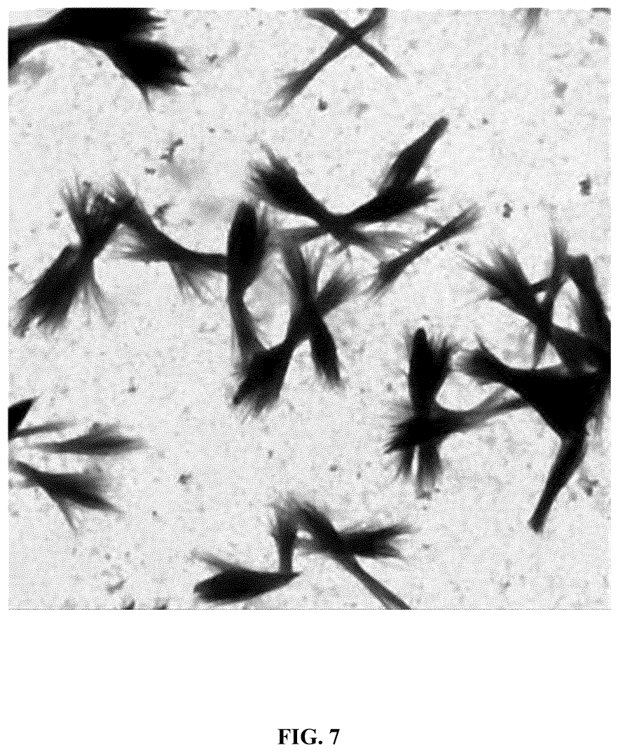

Result analysis: as shown in FIG. 5, picture a is an original protein, pictures b, c, dare the structural diagrams of rice protein with increasing eugenol concentration in turn; that is, in (3), when the amount of eugenol added is low, eugenol enters the rice protein to form small dispersed droplets, and no full hollow structure can be formed in the rice protein. After dialysis, it is found that the rice protein structure cannot be formed, as shown in pictures a and b in FIG. 6; when the concentration of eugenol is 1%, a large liquid cavity formed by eugenol can be observed in the rice protein. After dialysis, it is found that a complete cavity structure is formed in the rice protein, as shown in picture c of FIG. 6. However, when the concentration of eugenol is greater than 1%, it has little effect on the formation of the cavity structure of rice protein.

### Comparison Example 1

A method for preparing protein nanoparticle microspheres with adjustable hollow structure was detailed as follows:
(1) Structural unfolding of proteins: 0.1 g of rice protein powders were dispersed in ultrapure water in a mass-volume ratio of 1:100 g/mL. The pH of the rice protein solution was adjusted to 10.0 with 4 mol/L NaOH solution. The mixed solution was stirred completely for the structural unfolding of proteins, and centrifuged at 10,000 g for 10 min. The supernatant was collected and precipitant removed.
(2) Ion exchange: The pH of the supernatant obtained in (1) was adjusted using hydrogen type macroporous weakly acidic acrylic cation exchange resin to 7.0, and centrifuged at 10,000 g for 10 min. The precipitant was removed, and a uniform rice protein nanoparticle dispersion having an average particle size of 20 nm was obtained.
(3) Core-shell structure development: 1% of carvacrol (based on the volume of the rice protein nanoparticle dispersion) was added to the rice protein nanoparticle dispersion. The mixture was stirred completely, and centrifuged at 4,000 g for 10 min. The precipitant was removed, and a carvacrol-rice mixed solution was obtained.
(4) Hollow structure regulation: The carvacrol-rice mixed solution obtained in (3) was dialyzed in ultrapure water for 24 hours, and centrifuged at 10,000 g for 10 min. The precipitant was removed, and the upper dispersion was first cooled in a −20° C. freezer for 2 hours, and then stored overnight in a −80° C. freezer, freeze dried for 72 hours, to yield rice protein products.

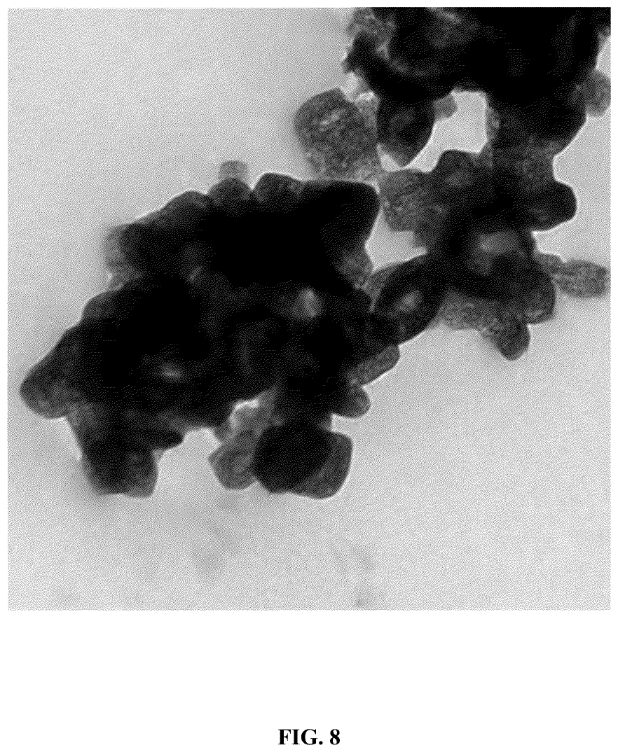

As shown in FIG. 7, the rice protein is in the shape of needles, is unable to form granular structure, let alone hollow structure.

### Comparison Example 2

A method for preparing protein nanoparticle microspheres with adjustable hollow structure was detailed as follows:
(1) Structural unfolding of proteins: 0.1 g of rice protein powders were dispersed in ultrapure water in a mass-volume ratio of 1:100 g/mL. The pH of the rice protein solution was adjusted to 10.0 with 4 mol/L NaOH solution. The mixed solution was stirred completely for the structural unfolding of proteins, and centrifuged at 10,000 g for 10 min. The supernatant was collected and precipitant removed.
(2) Ion exchange: The pH of the supernatant obtained in (1) was adjusted using hydrogen type macroporous weakly acidic acrylic cation exchange resin to 7.0, and centrifuged at 10,000 g for 10 min. The precipitant was removed, and a uniform rice protein nanoparticle dispersion having an average particle size of 20 nm was obtained.
(3) Core-shell structure development: 1% of thymol (based on the volume of the rice protein nanoparticle dispersion) was added to the rice protein nanoparticle dispersion. The mixture was stirred completely, and centrifuged at 4,000 g for 10 min. The precipitant was removed, and a thymol-rice mixed solution was obtained.
(4) Hollow structure regulation: The thymol-rice mixed solution obtained in (3) was dialyzed in ultrapure water for 24 hours, and centrifuged at 10,000 g for 10 min. The precipitant was removed, and the upper dispersion was first cooled in a −20° C. freezer for 2 hours, and then stored overnight in a −80° C. freezer, freeze dried for 72 hours, to yield rice protein products.

As shown in FIG. 8, the rice protein is in the shape of needles, is unable to form granular structure, let alone hollow structure.

It will be obvious to those skilled in the art that changes and modifications may be made, and therefore, the aim in the appended claims is to cover all such changes and modifications.

What is claimed is:

1. A method for preparing protein nanoparticle microspheres, the method comprising:
1) dispersing a protein powder in water, adjusting a pH of a solution of the protein powder to between 10.0 and 12.0, stirring, centrifuging, and collecting a first upper dispersion;
2) adjusting the first upper dispersion in 1) to be neutral using a cation exchange resin, centrifuging, to yield a protein nanoparticle dispersion;
3) adding eugenol to the protein nanoparticle dispersion, stirring, centrifuging, and removing a precipitant, to yield a core-shell nanoprotein dispersion comprising a eugenol core and a protein shell; and
4) adding the core-shell nanoprotein dispersion to ultrapure water, dialyzing, centrifuging, collecting a second upper dispersion, freeze-drying the second upper dispersion, to yield protein nanoparticle microspheres.

2. The method of claim 1, wherein following 2) and before adding eugenol to the protein nanoparticle dispersion, the method further comprises: adding sodium chloride to the protein nanoparticle dispersion, to induce an aggregation of protein nanoparticles; centrifuging the protein nanoparticle dispersion comprising aggregated protein nanoparticles, and removing the precipitant, to yield a dimension-enlarged protein nanoparticle dispersion.

3. The method of claim 2, wherein a final concentration of sodium chloride in the protein nanoparticle dispersion is 5-10 μmol/L.

4. The method of claim 1, wherein in 3), the eugenol accounts for 1% by volume of the protein nanoparticle dispersion.

5. The method of claim 2, wherein in 3), the eugenol accounts for 1% by volume of the protein nanoparticle dispersion.

6. The method of claim 1, wherein a mass-volume ratio of the protein powder to the eugenol is 0.1:100 g/μL.

7. The method of claim 2, wherein a mass-volume ratio of the protein powder to the eugenol is 0.1:100 g/μL.

8. The method of claim 1, wherein in 4), a dialysis time is 12-24 hours.

9. The method of claim 2, wherein in 4), a dialysis time is 12-24 hours.

10. The method of claim 1, wherein in 1), a mass-volume ratio of the protein powder to the water is between 1:50 and 1:100 g/mL.

11. The method of claim 2, wherein in 1), a mass-volume ratio of the protein powder to the water is between 1:50 and 1:100 g/mL.

12. The method of claim 1, wherein in 4), when a dialysis time is 24 hours, full-hollow protein nanoparticle microspheres are obtained; when a dialysis time is 18 hours, semi-hollow protein nanoparticle microspheres are obtained; and when a dialysis time is 12 hours, solid-like protein nanoparticle microspheres are obtained.
